(19) United States
(12) Patent Application Publication
(10) Pub. No.: US 2020/0016052 A1
(43) Pub. Date: Jan. 16, 2020

(54) ORGANIC UV BLOCKER-SUPPORTED SILICA AEROGEL COMPOSITE

(71) Applicant: ASTIN CO., LTD., Daejeon (KR)

(72) Inventors: Chul Hwan KIM, Daejeon (KR); Yeon Ju PARK, Yongin-si (KR); Hye Mun KIM, Daejeon (KR); Ja Eun LEE, Daejeon (KR)

(73) Assignee: ASTIN CO., LTD., Daejeon (KR)

(21) Appl. No.: 16/335,569

(22) PCT Filed: Sep. 18, 2017
(86) PCT No.: PCT/KR2017/010152

§ 371 Date: Mar. 21, 2019

(30) Foreign Application Priority Data
Sep. 23, 2016 (KR) 10-2016-0122494

(51) Int. Cl. A61K 8/25; A61K 9/12; A61Q 17/04; A61K 8/44; A61K 8/362; A61K 8/34

(52) U.S. Cl. CPC A61K 8/25; A61K 9/12; A61Q 17/04; A61K 8/345; A61K 8/362; A61K 8/347; A61K 8/44

(57) ABSTRACT

The present invention relates to a silica aerogel composite comprising a hydrophilically surface-modified hydrophobic silica aerogel and an organic UV blocker supported in pores of the silica aerogel.

The silica aerogel composite of the present invention causes no skin irritation since the organic UV blocker inducing skin irritation is stably supported inside the silica aerogel to minimize the contact with the skin. Also, the silica aerogel composite is high in the organic UV blocker content per unit weight, and exhibits an excellent blocking effect against both UVA and UVB compared with conventional UV blockers.

### TECHNICAL FIELD

The present invention relates to a silica aerogel composite including a hydrophilically surface-modified hydrophobic silica aerogel and an organic UV blocker supported in pores of the silica aerogel.

### BACKGROUND ART

Sun light is largely divided into visible light, infrared light and ultraviolet light depending on the length of wavelength. Among these, ultraviolet light, light shorter than purple of visible light, is referred as UV and divided into three regions. UVA is a primary cause of skin blackening, and produces wrinkles and accelerates aging by penetrating into the dermis of the skin and inhibiting with a regenerative function of the skin. UVB has a shorter wavelength than UVA and does not penetrate into the dermis, but causes erythema and burns, thickens the stratum corneum, dries the skin to roughen the skin surface, and produces melanin pigment to produce discoloration, freckles, age spots and the like. In order to protect the skin from such UV, using a UV blocker having a proper UV blocking index is essential.

A UV blocker is largely divided into an organic UV blocker and an inorganic UV blocker. An inorganic UV blocker is also referred to as a physical blocker, and blocks UV by physically scattering UV reaching the skin without permeating into the skin. An organic UV blocker is also referred to as a chemical UV blocker, and blocks UV by absorbing energy of reaching UV.

Among these, an organic UV blocker has advantages of no white cast, and excellent spreadability and feeling of use, and the use is gradually increasing. However, the organic UV blocker penetrates into the skin to function, and has disadvantages in that the blocker itself causes irritation, or products by a photoreaction causes irritation to cause irritant contact dermatitis to the sensitive skin.

In order to overcome the above-described problems, attempts to minimize the contact with the skin by supporting an organic UV blocker in a support have been continuously made. As one example, Korean Patent No. 1175623 discloses a complex pigment in which an organic UV blocker is supported in hollow spherical silica. However, the complex pigment may be used only in a dry powder formulation, and when used in a water in oil (W/O) or oil in water (O/W) emulsion formulation, the organic UV blocker may readily come out of the support, and the effect of mitigating skin irritation may not be secured.

In view of the above, development of a new composite for UV blocking stable in a formulation while having an excellent UV blocking effect and causing no skin irritation has been required.

### PRIOR ART DOCUMENTS

### Patent Documents

Korean Patent No. 1175623, Method for preparing organic UV blocker-supported hollow spherical silica complex pigment and a cosmetic composition for UV blocking containing the same

### DISCLOSURE

### Technical Problem

In view of the above, the inventors of the present invention have prepared a composite using a hydrophobic silica aerogel having its surface modified to be hydrophilic as a support of an organic UV blocker, have identified that the composite exhibits an excellent UV blocking effect and is hypoallergenic, and have completed the present invention.

Accordingly, an aspect of the present invention provides an organic UV blocker-supported silica aerogel composite, and a cosmetic composition for UV blocking including the same.

### Technical Solution

According to an aspect of the present invention, there is provided a silica aerogel composite including a hydrophilically surface-modified hydrophobic silica aerogel and an organic UV blocker supported in pores of the silica aerogel.

### Advantageous Effects

A silica aerogel composite of the present invention causes no skin irritation since an organic UV blocker inducing skin irritation is stably supported inside the silica aerogel to minimize a contact with the skin. In addition, the silica aerogel composite is capable of securing an excellent UV blocking effect even in a small amount by having a high organic UV blocker content per unit weight, and exhibits an excellent blocking effect against both UVA and UVB compared with conventional UV blockers.

### DESCRIPTION OF DRAWINGS

FIG. 1 is a photograph comparing cases of (a) dispersing a hydrophobic silica aerogel before surface modification into distilled water and (b) dispersing a hydrophobic silica aerogel after surface modification into distilled water.

FIG. 2 is a thermogravimetric analysis (TGA) graph of a silica aerogel before supporting an organic UV blocker (a) and an organic UV blocker-supported silica aerogel composite (b).

FIG. 3 is a graph showing an SPF index of a cosmetic composition for UV blocking prepared in Preparation Example 2.

FIG. 4 is a graph showing a PA index of a cosmetic composition for UV blocking prepared in Preparation Example 2.

### BEST MODE

Hereinafter, the present invention will be described in detail so that those skilled in the art readily implement the present invention. However, the present invention may be embodied in various different forms, and is not limited to embodiments described herein.

Silica Aerogel Composite

The present invention provides a silica aerogel composite including a hydrophilically surface-modified hydrophobic silica aerogel and an organic UV blocker supported in pores of the silica aerogel.

An aerogel is a substance obtained by substituting a liquid component of a gel with the air through supercritical drying, and is an ultra-light, ultra-porous and high specific surface area material having porosity of 85% to 99.9% and a pore size of approximately 1 nm to 100 nm. Particularly, a silica aerogel prepared from silica gel has excellent insulating properties, sound-proofing properties and low dielectric properties, and therefore, has received attention as a material of transparent insulation materials, environmental-friendly high-temperature-type insulation materials, sound-proofing materials, extremely low dielectric thin films for highly integrated devices, catalysts, catalyst supports and the like. Meanwhile, due to its adsorption ability obtained from a porous structure, a silica aerogel is used for adsorbing skin wastes in the field of cosmetics.

Based on the fact that a silica aerogel has high porosity and pore volume, the inventors of the present invention utilize a silica aerogel as a support of an organic UV blocker.

According to the present invention, an organic UV blocker inducing skin irritation is supported inside a hydrophobic silica aerogel and is not in direct contact with the skin, and therefore, skin irritation may be minimized, and by the hydrophobic silica aerogel having its surface modified to be hydrophilic, the organic UV blocker does not leak out of the support in the formulation. In addition, the hydrophobic silica aerogel has high porosity and pore volume compared to existing supports, and therefore, may increase an amount of organic UV blocker filling per unit volume. Accordingly, the silica aerogel composite according to the present invention exhibits an excellent UV blocking ability even in a small amount.

Particularly, the silica aerogel composite according to the present invention has a structure in which silica, an air layer and an organic UV blocker are present in multiple layers, and, due to such structural features, exhibits a high UV blocking ability. In other words, the silica, the air and the organic UV blocker each have a different refractive index, and light scattering occurs several times when irradiating light on the silica aerogel composite of the present invention. Accordingly, a significantly enhanced UV blocking ability is obtained compared to when a support is not used or a different support is used.

The silica aerogel used in the present invention is preferably a hydrophobic silica aerogel having its surface modified to be hydrophilic. Herein, the hydrophobic silica aerogel may be purchased commercially or directly prepared to be used.

A method for preparing the hydrophobic silica aerogel is not particularly limited in the present invention, and methods commonly used in the art may be used. As one example, the hydrophobic silica aerogel may be prepared by diluting a silica precursor such as water glass or tetraethoxysilane (TEOS), adding 4.3 N acetic acid thereto to prepare a silica gel, then solidifying the result, removing salts from the solidified gel, extracting the result until the water content becomes 10% or less, and enclosing the result in an autoclave with a sufficient amount of alcohol and supercritical drying the result under pressure to remove a liquid component inside the wet gel.

The hydrophobic silica aerogel is used as a support of an organic UV blocker after going through a surface treatment process so as to have a hydrophilic functional group on the particle surface.

An organic UV blocker is mostly a hydrophobic molecule that is not well-dissolved in a water phase and is well-dissolved in an oil phase component, and, through hydrophobic interaction with a hydrophobic silica aerogel, may penetrate into pores of the aerogel. However, when preparing a composite using a hydrophobic silica aerogel as it is without surface treatment as a support of an organic UV blocker, the composite needs to be dispersed into an oil phase when preparing a cosmetic composition, and in this case, the organic UV blocker may be eluted to the outside without maintaining a supported state. As a result, a goal of minimizing a contact of the organic UV blocker with the skin is not accomplished.

The present invention resolves a problem as above by using a hydrophobic silica aerogel having a hydrophilic functional group such as a hydroxyl group (—OH) on the surface while maintaining hydrophobicity inside. The silica aerogel has excellent dispersibility in a water phase by having a hydrophilic functional group on an outer circumference while being capable of hydrophobic interaction with a hydrophobic organic UV blocker since the inside is hydrophobic. Meanwhile, the hydrophobic organic UV blocker is not well-dissolved in a water phase, and therefore, in the silica aerogel composite having an organic UV blocker supported inside the silica aerogel, the organic UV blocker may stably maintain a supported state without being eluted when dispersing the silica aerogel composite into a water phase.

In the present invention, a method of surface modifying the hydrophobic silica aerogel is not particularly limited, and methods generally used in the art such as an aqueous acidic solution treatment or an ultrasonic treatment may be used.

Preferably, the hydrophilically surface-modified hydrophobic silica aerogel may be prepared by dispersing a hydrophobic silica aerogel into an aqueous C1 to C4 lower alcohol solution, and then heat treating the result.

Herein, the C1 to C4 lower alcohol may be methanol, ethanol, propanol, isopropanol or butanol, and ethanol is preferably used. Water and alcohol are preferably mixed in a ratio of 1:1 to 4:1 in the aqueous lower alcohol solution. In addition, the heat treatment process is preferably performed at 200° C. to 400° C., and preferably at 300° C. to 400° C.

Through the above-described method, the hydrophobic silica aerogel may be modified to be hydrophilic only on the outer circumference of the particle while maintaining hydrophobicity inside the particle.

The surface-modified hydrophobic silica aerogel preferably has porosity of 90% or greater in order to maximize an organic UV blocker content.

The porosity means a ratio of volume occupied by pores with respect to a total volume of a porous material, and may be represented by, when a dry weight of the porous material is w1, the weight in water is w2 and the saturated weight (weight when filling the pores with water) is w3, apparent porosity={(w3−w1)/(w3−w2)}1×100.

A pore diameter of the surface-modified hydrophobic silica aerogel is not particularly limited, but is preferably from 2 nm to 10 nm. When the pore diameter is less than 2 nm, the organic UV blocker is difficult to penetrate into the pores causing a problem of decreasing the amount of filling, and when the pore diameter is greater than 10 nm, the organic UV blocker readily leaks out of the pores, and therefore, the pore diameter is properly adjusted in the above-mentioned range.

The surface-modified hydrophobic silica aerogel preferably has a specific surface area of 250 m²/g to 400 m²/g, and more preferably 300 m²/g to 350 m²/g. Such a wide specific surface area enables interaction with the organic UV blocker molecules, and therefore, the silica aerogel composite according to the present invention may stably maintain the organic UV blocker-supported structure.

A particle size of the surface-modified hydrophobic silica aerogel used in the present invention is not particularly limited, but is preferably from 2 μm to 15 μm, and more preferably from 5 μm to 10 μm for uniform distribution when added to a cosmetic composition and giving smooth feeling of use when applied to the skin. The hydrophobic silica aerogel having its surface modified to be hydrophilic satisfying the particle size may be obtained by surface treating a hydrophobic silica aerogel having a particle size of 30 μm to 200 μm, and then dry or wet grinding the result using a proper method such as a ball mill or a rod mill. Preferably, a method of dispersing the surface-treated hydrophobic silica aerogel into distilled water, and then wet grinding the result through a ball mill process is used.

The organic UV blocker used in the present invention is a hydrophobic organic material, and is not particularly limited as long as it is effective in blocking UVA or UVB, and materials generally used in the art may be used. Nonlimiting examples of the organic UV blocker may include one or more types selected from the group consisting of ethylhexyl methoxycinnamate, ethylhexyl silicate, ethylhexyl salicylate, ethylhexyl triazone, octyldimethyl para-aminobenzoic acid, butyl methoxydibenzoylmethane, isoamyl-p-methoxycinnamate, octyl salicylate, octocrylene, butyl methoxydibenzoylmethane, oxybenzone, octyl triazone, menthyl anthranilate, 3,4-methylbenzylidene camphor, bis-ethylhexyloxyphenol methoxyphenyl triazine, homosalate, phenylbenzimidazole sulfonic acid and diethylamino hydroxybenzoyl hexyl benzoate.

Preferably, the organic UV blocker uses a mixture of octocrylene and butyl methoxydibenzoylmethane, and herein, the octocrylene and the butyl methoxydibenzoylmethane are preferably included in a ratio of 2:1 to 4:1.

As a method of filling the silica aerogel with the organic UV blocker, a solvent method, an initial wetting method, a simple mixing method without using any solvent or mixing aids, or the like may be used.

The solvent method means a method of treating a silica aerogel with an organic UV blocker-containing solution, and then removing the solvent.

The initial wetting method is also referred to as capillary impregnation or dry impregnation, and, as a method of impregnating an organic UV blocker by a capillary action, is a method of treating a silica aerogel with a concentrated solution including an organic UV blocker.

According to one preferred embodiment of the present invention, an organic UV blocker is introduced while stirring a solution in which a surface-treated hydrophobic silica aerogel is dispersed into distilled water using a homogenizer, and the organic UV blocker is supported by stirring while rapidly cooling the mixture.

The silica aerogel composite prepared through such a supporting method includes the organic UV blocker in 50% by weight to 75% by weight. By having such a high amount of filling, the silica aerogel composite according to the present invention exhibits an excellent UV blocking ability even in a small amount compared to existing UV blocker-supported composites.

Cosmetic Composition for UV Blocking

The silica aerogel composite according to the present invention may be used as a raw material of a cosmetic composition for UV blocking.

Herein, the cosmetic composition for UV blocking includes the silica aerogel composite in 1% by weight to 30% by weight and more preferably in 4% by weight to 20% by weight as a UV blocker. When the silica aerogel composite content is less than 1% by weight, a sufficient UV blocking effect is difficult to secure, and when the content is greater than 30% by weight, the organic UV blocker may cause skin irritation due to an increased chance of being in contact with the skin, and therefore, the content is properly adjusted in the above-mentioned range.

The cosmetic composition for UV blocking may further include an inorganic UV blocker in order to increase a UV blocking effect.

Types of the inorganic UV blocker are not particularly limited, and materials commonly used in the art may be used. Specifically, one or more types selected from the group consisting of titanium dioxide, iron oxide and zinc oxide may be used.

In addition, the cosmetic composition for UV blocking according to the present invention may further include additives known in the art.

Nonlimiting examples of the usable additives may include a moisturizer, a fatty acid, a thickener, a preservative, a pH adjusting agent, an antioxidant, a pigment, an emulsifier, a chelating agent, a conditioning agent, a perfume and the like.

As the moisturizer, one type selected from the group consisting of erythritol, xylitol, maltitol, propylene glycol, sorbitol, polyglycerin, polyethylene glycol, 1,2-pentanediol, 1,3-butylene glycol, amino acid, sodium lactate, sodium pyrrolidone carboxylate, xyloglucan, quince seed, carrageenan, pectin, mannan, curdlan, galactan, dermatan sulfate, glycogen, keratan sulfate, chondroitin, mucoitin sulfate, kerato sulfate, locust bean gum, succinoglycan, calonym acid, hyaluronic acid, heparin sulfate, sodium hyaluronate, collagen, mucopolysaccharide, chondroitin sulfate, dimethyl polysiloxane, methylphenyl siloxane, culture supernatant of lactic acid bacteria or bifidobacterial, and combinations thereof may be included.

As the fatty acid, one type of fatty acid selected from the group consisting of lauric acid, myristic acid, palmitic acid, stearic acid, oleic acid, linoleic acid and mixtures thereof may be included.

The thickener provides proper viscosity when used as a cosmetic composition to enhance feeling of use, and one type selected from the group consisting of sodium alginate, xanthan gum, aluminum silicate, quince seed extract, Arabic gum, hydroxyethyl guar gum, carboxymethyl guar gum, guar gum, dextran, tragacanth gum, cellulose, hydroxypropyl cellulose, methylhydroxypropyl cellulose, methyl cellulose, carboxymethyl cellulose, hydroxyethyl cellulose, carboxyvinyl polymers, polyvinyl alcohol, polyvinylpyrrolidone, carbomers, acrylate copolymers, acrylate/C10-30 alkyl acrylate cross polymers and combinations thereof may be used.

As the preservative, parabens such as methyl paraben or propyl paraben, phenoxyethanol, octanediol, hexanediol, benzoates, salicylates, sorbates, dihydroacetates, paraoxybenzoic acid esters, 2,4,4-trichloro-2-hydroxydiphenyl ether, 3,3,4-trichlorocarbanilide, benzalkonium chloride, hinokitiol, resorcin and the like may be used.

As the pH adjusting agent, sodium hydroxide, triethanolamine, citric acid, sodium citrate, boric acid, borax, potassium hydrogen phosphate and the like may be used.

As the antioxidant, dibutylhydroxytoluene, butylhydroxyanisole, propyl gallate, ascorbic acid and the like may be used.

The pigment includes an extender pigment, a white pigment, a coloring pigment, a pearlescent pigment, a metal powder, an organic powder and the like. Examples of the extender pigment may include talc, mica, kaolin, calcium carbonate, alumina, barium silicate, zeolite, muscovite, magnesium carbonate, barium sulfate and the like, examples of the white pigment may include titanium dioxide, zinc oxide and the like, examples of the coloring pigment may include bengala, yellow iron oxide, black iron oxide, chromium oxide, navy blue, Prussian blue, carbon black and the like, examples of the pearlescent pigment may include titanium dioxide, mica titanium, iron titanate, titanium oxide-covered mica, silica, tin oxide, ferric ferrocyanide and the like, examples of the metal powder may include gold, silver, copper, palladium, platinum and the like, and examples of the organic powder may include polymethyl methacrylate, nylon, cellulose, starch and the like.

Examples of the emulsifier may include lecithin, polyglyceryl-3-methyl glucose distearate, cetearyl alcohol, glyceryl monostearate and the like.

As the chelating agent, disodium EDTA, tetrasodium EDTA, phosphoric acid, citric acid, ascorbic acid, succinic acid, gluconic acid, sodium polyphosphate, sodium metaphosphoate and the like may be used.

As the conditioning agent, phenyl trimethicone, cyclomethicone, glycereth-26, glycerin, a sodium PCA solution, sorbitol, Shea butter, stearyl stearate, stearyl heptanoate, vegetable squalane, glyceryl rosinate, sodium polyacrylate, C12-15 alcohol benzoate, aspartic acid and the like may be used.

As the perfume, a natural perfume, a combined perfume and the like may be used.

The cosmetic composition for UV blocking according to the present invention may be prepared in an oil in water (O/W) or water in oil (W/O) emulsion type. Herein, the silica aerogel composite of the present invention is dispersed into the water phase. By the surface of the silica aerogel composite including a hydrophilic group such as a hydroxyl group, the silica aerogel composite is more readily dispersed uniformly into the water phase than the oil phase, and the hydrophobic organic UV blocker supported inside does not come out, and therefore, dispersing into the water phase is preferred.

The cosmetic composition for UV blocking according to the present invention may be prepared in any formulation commonly prepared in the art. For example, the cosmetic composition for UV blocking according to the present invention may be formulated into a solution suspension, an emulsion, a paste, a gel, a cream, a lotion, a powder, an oil, a powder foundation, an emulsion foundation, a wax foundation, a spray and the like, however, the formulation is not limited thereto. In more detail, the cosmetic composition for UV blocking according to the present invention may be prepared into a formulation of a sun cream, a softening face lotion, an astringent face lotion, a nourishing face lotion, a nourishing cream, an essence, an eye cream, a pack, a spray or a powder.

When the formulation of the present invention is a solution or an emulsion, a solvent, a solvating agent or an emulsifier is used as a support component, and examples thereof may include water, ethanol, isopropanol, ethyl carbonate, ethyl acetate, benzyl alcohol, benzyl benzoate, propylene glycol, 1,3-butylglycol oil, glycerol aliphatic ester, polyethylene glycol or sorbitan fatty acid ester.

When the formulation of the present invention is a suspension, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth or the like may be used as a support component.

When the formulation of the present invention is a paste, a cream or a gel, animal oil, vegetable oil, wax, paraffin, starch, tragacanth, cellulose derivatives, polyethylene glycol, silicon, bentonite, silica, talc, zinc oxide or the like may be used as a support component.

When the formulation of the present invention is a powder or a spray, lactose, talc, silica, aluminum hydroxide, calcium silicate or polyamide powder may be used as a support component, and when the formulation is a spray in particular, a propellant such as chlorofluorohydrocarbon, propane/butane or dimethyl ether may be included.

Herein, the present invention will be described in more detail with reference to examples and drawings. However, it is obvious to those skilled in the art that the present invention is not limited to the following examples, and various changes or modifications may be made within the scope of technological ideas of the present invention.

### EXAMPLE

### Preparation Example 1: Preparation of Silica Aerogel Composite

### (1) Preparation of Surface-Modified Hydrophobic Silica Aerogel

A surface of a powder-type hydrophobic silica aerogel (RAGEL-P, average particle size 50 μm) manufactured by REM Tech. was modified to be hydrophilic using the following method.

A hydrophobic silica aerogel (20 g) was added to a mixed solvent of distilled water (100 g) and ethanol (50 g), and mixed. The mixture was poured into a SUS plate and introduced to a furnace, and then heat treated for 2 hours at 350° C. to modify a surface of the hydrophobic silica aerogel to be hydrophilic.

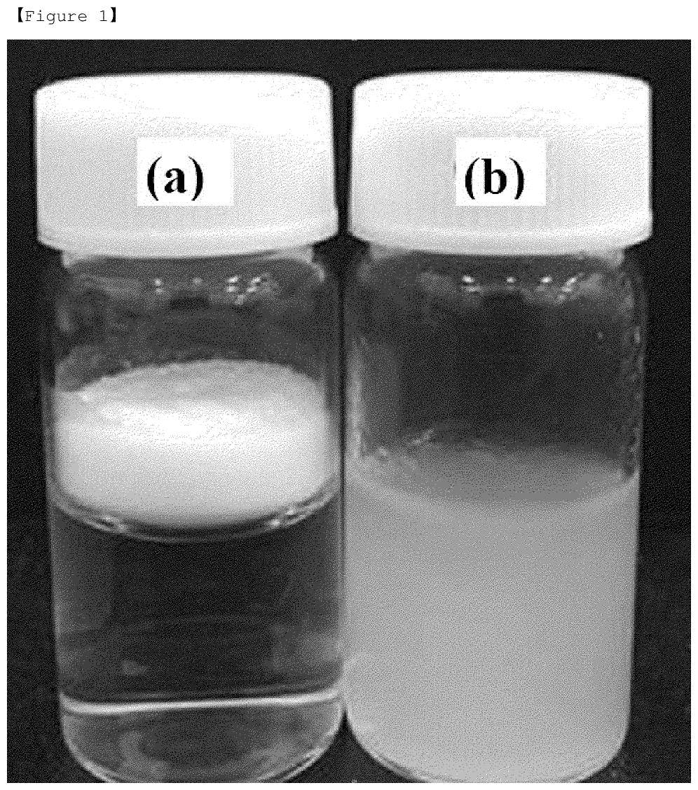

FIG. 1 is a photograph comparing cases of (a) dispersing the aerogel before surface modification into distilled water and (b) dispersing the aerogel after surface modification into distilled water. It was identified that the hydrophobic silica aerogel after surface modification was evenly dispersed into water whereas the hydrophobic silica aerogel before surface modification had a layer separation without being dispersed into water.

### (2) Preparation of Silica Aerogel Composite

After introducing distilled water (73.33 g) to the surface-modified hydrophobic silica aerogel (20 g) prepared in (1), the result was mixed and then ball milled for 1 hour at 200 rpm using a 0.5 cm zirconia ball so that the surface-modified hydrophobic silica aerogel has an average particle size of 5 μm to 10 μm.

Octocrylene (15 g) and avobenzone (butyl methoxydibenzoylmethane, 5 g) were well-mixed, and heated to 70° C. in an oven to dissolve the avobenzone in the octocrylene.

An aqueous solution (46.66 g) including the ground silica aerogel (10 g) was introduced to a container, and stirred at 400 rpm using a homonixer, and after taking out the mixture of octocrylene and avobenzone from the oven and introducing the mixture thereto, the result was stirred for 10 minutes to well-mix the organic UV blocker and the silica aerogel. Then, the result was stirred for 10 minutes while quickly cooling using ice water to prepare an organic UV blocker-supported silica aerogel composite.

### Experimental Example 1: Measurement of Supported Amount of Organic UV Blocker

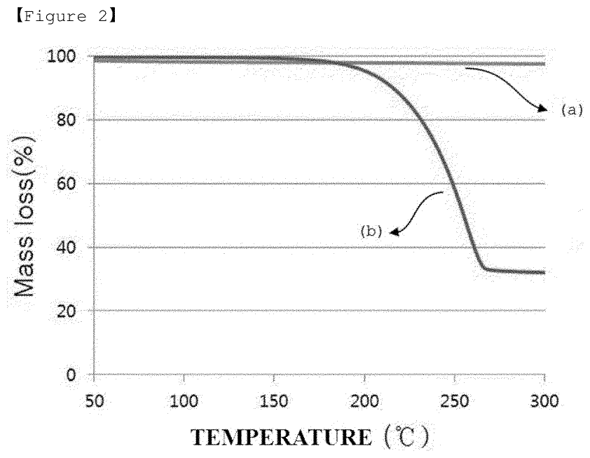

In order to identity the supported amount of organic UV blocker in the silica aerogel composite prepared in Preparation Example 1, a thermogravimetric analysis (TGA) was performed using a thermal analyzer (Mettler-Toledo TGA/DSC 1), and the results are shown in FIG. 2.

When referring to FIG. 2, it was identified that the silica aerogel before supporting the organic UV blocker (a) had no weight changes, whereas the organic UV blocker-supported silica aerogel composite (b) had a mass loss of 66% at 250° C. or higher. From this, it was identified that the silica aerogel composite of Preparation Example 1 had an organic UV blocker supported therein, and the content was 66% by weight of the total weight of the composite.

### Preparation Example 2: Preparation of Cosmetic Composition for UV Blocking

### (1) Preparation of Examples 1 to 3

Cosmetic compositions for UV blocking of Examples 1 to 3 were prepared in the compositions of the following Table 1 (unit: % by weight).

TABLE 1

| | Name of Raw | | | | |
|---|---|---|---|---|---|
| | Material | Role | Example 1 | Example 2 | Example 3 |
| A | Distilled Water | Solvent | To 100 | To 100 | To 100 |
| | Silica Aerogel | UV Blocker | 6.07 | 9.1 | 13.7 |
| | composite | | | | |
| | EDTA-2Na | Chelating | 0.02 | 0.02 | 0.02 |
| | | Agent | | | |
| | Phenoxyethanol | Preservative | 0.5 | 0.5 | 0.5 |
| | Danisol-M | | 0.2 | 0.2 | 0.2 |
| | 1,3-Butylene | Moisturizer | 7 | 7 | 7 |
| | Glycol | | | | |
| B | 1% Carbopol 940 | Thickener | 10 | 10 | 10 |
| C | Danisol-P | Preservative | 0.1 | 0.1 | 0.1 |
| | TegoCare 450 | Emulsifier | 1.2 | 1.2 | 1.2 |
| | Lanette-O | | 1 | 1 | 1 |
| | Arlacel 165 | | 1.1 | 1.1 | 1.1 |
| | DC 556 | Conditioning | 2 | 2 | 2 |
| | DC 345 | Agent | 8 | 8 | 8 |

Water phase components of A were mixed and heated to 80° C., and the thickener of B was introduced thereto to prepare a water phase portion. Oil phase components of C were mixed and heated to 80° C. to prepare an oil phase portion. The oil phase portion was added to the water phase portion, and the result was stirred for 5 minutes at 3000 rpm using a homogenizer to prepare an oil in water-type cosmetic composition for UV blocking.

### (2) Preparation of Comparative Examples 1 to 6

Cosmetic compositions for UV blocking of Comparative Examples 1 to 6 were prepared using the preparation method of Examples 1 to 3 in the compositions of the following Tables 2 and 3 (unit: % by weight) while varying only the UV blocker type.

As the UV blocker, a mixture of octocrylene:avobenzone=3:1 was used as it is (Comparative Examples 1, 3 and 5), or a composite prepared by supporting this in SILINOS-350, a spherical silica powder, was used (Comparative Examples 2, 4 and 6). Herein, the supported amount of the UV blocker in the SILINOS-350 composite was 50% of the total weight of the composite.

TABLE 2

| | | | | Compar- |
|---|---|---|---|---|
| | | | Comparative | ative |
| | Name of Raw Material | Role | Example 1 | Example 2 |
| A | Distilled Water | Solvent | To 100 | To 100 |
| | Octocrylene:Avobenzone = | UV blocker | 4 | — |
| | 3:1 | | | |
| | SILINOS-350 Composite | | — | 8 |
| | EDTA-2Na | Chelating | 0.02 | 0.02 |
| | | Agent | | |
| | Phenoxyethanol | Preservative | 0.5 | 0.5 |
| | Danisol-M | | 0.2 | 0.2 |
| | 1,3-Butylene Glycol | Moisturizer | 7 | 7 |
| B | 1% Carbopol 940 | Thickener | 10 | 10 |
| C | Danisol-P | Preservative | 0.1 | 0.1 |
| | TegoCare 450 | Emulsifier | 1.2 | 1.2 |
| | Lanette-O | | 1 | 1 |
| | Arlacel 165 | | 1.1 | 1.1 |
| | DC 556 | Conditioning | 2 | 2 |
| | DC 345 | agent | 8 | 8 |
| | Total Sum | | 100 | 100 |

TABLE 3

| | Name of Raw | Comparative | Comparative | Comparative | Comparative |
|---|---|---|---|---|---|
| | Material | Example 3 | Example 4 | Example 5 | Example 6 |
| A | Distilled Water | To 100 | To 100 | To 100 | To 100 |
| | Octocrylene:Avobenzone = | 6 | — | 9 | — |
| | 3:1 | | | | |
| | SILINOS-350 | — | 12 | — | 18 |
| | Composite | | | | |
| | EDTA-2Na | 0.02 | 0.02 | 0.02 | 0.02 |
| | Phenoxyethanol | 0.5 | 0.5 | 0.5 | 0.5 |
| | Danisol-M | 0.2 | 0.2 | 0.2 | 0.2 |
| | 1,3-Butylene Glycol | 7 | 7 | 7 | 7 |
| B | 1% Carbopol 940 | 10 | 10 | 10 | 10 |
| C | Danisol-P | 0.1 | 0.1 | 0.1 | 0.1 |
| | TegoCare 450 | 1.2 | 1.2 | 1.2 | 1.2 |
| | Lanette-O | 1 | 1 | 1 | 1 |
| | Arlacel 165 | 1.1 | 1.1 | 1.1 | 1.1 |
| | DC 556 | 2 | 2 | 2 | 2 |
| | DC 345 | 8 | 8 | 8 | 8 |
| | Total Sum | 100 | 100 | 100 | 100 |

### Experimental Example 1: Measurement or UV Blocking Index

UV blocking indices (SPF, PA) of the cosmetic compositions for UV blocking prepared in Preparation Example 2 were measured.

SPF (sun protection factor) is an index representing a UVB blocking effect, and may be represented by the following Mathematical Formula 1.

SPF=(MED of area where sample is applied)/(MED of area where sample is not applied) [Mathematical Formula 1]

The MED (minimal erythemal dose) means minimum UV irradiation causing skin erythema.

PA (protection grade of UVA) is an index representing a UVA blocking effect, and may be represented by the following Mathematical Formula 2.

PFA=(MPPD of area where sample is applied)/(MPPD of area where sample is not applied) [Mathematical Formula 2]

The MPPD (minimal persistent pigment darkening dose) means minimum UV irradiation at which faint blackening is recognized.

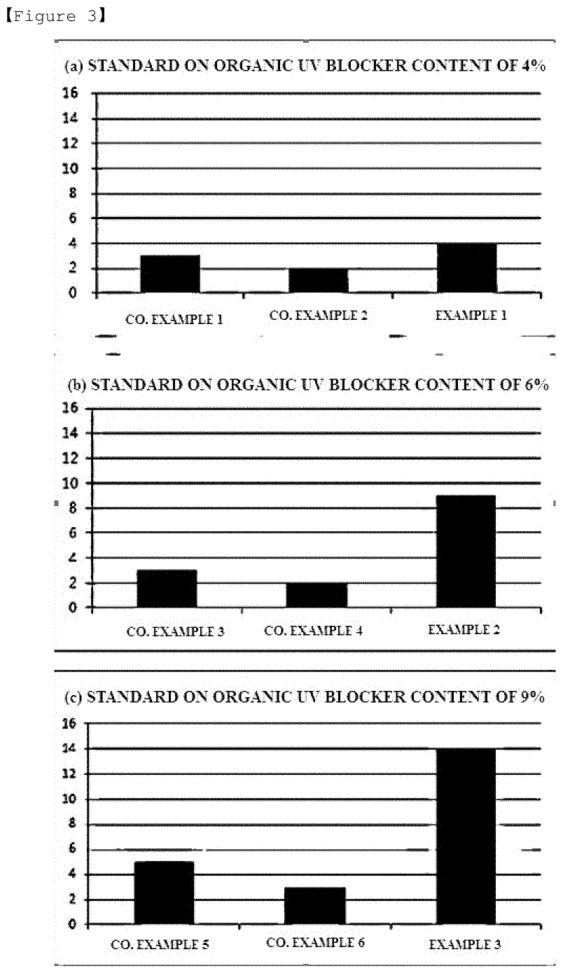

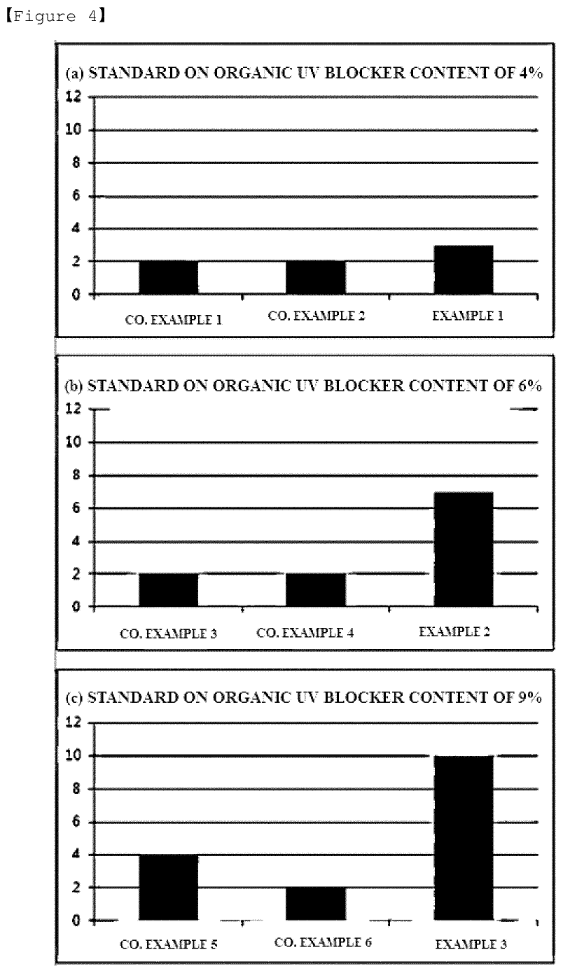

2 mg/cm² of each of the cosmetic compositions for UV blocking of the examples and the comparative examples was applied to the skin of 10 adult males and females, and after irradiating UV using a solar simulator lamp, average MED and MPPD values for group 1 of 5 subjects were measured, and the results are shown in FIG. 3 (SPF index) and FIG. 4 (PA index).

When referring to FIG. 3 and FIG. 4, it was identified that, when using octocrylene and avobenzone as it is without supporting in a support or using SILINOS-350 as a support, the UV blocking index was not much different even when increasing the organic UV blocker content in the composition.

However, the silica aerogel composite of the present invention exhibited a high UV blocking index of maximally 5 times in the same organic UV blocker content compared to the comparative examples, and particularly, it was identified that, when the organic UV blocker content was 6% or higher, the UV blocking index significantly increased, and an excellent UV blocking effect was obtained even in a small amount.

Such a high UV blocking ability may be considered to be obtained from a unique structure of the silica aerogel composite. In other words, the silica aerogel composite of the present invention has a structure in which silica, an air layer and an organic UV blocker are present in multiple layers, and light scattering occurs several times since each of these layers has a different refractive index, which brings a high UV blocking effect.

### Experimental Example 2: Test on Skin Irritation

Skin irritation of the cosmetic compositions for UV blocking of Example 3 and Comparative Examples 5 and 6 was tested by conducting a patch test on 10 healthy adult males and females who have not had a hypersensitivity reaction to skin irritation in the past. The test method is as follows. After preparing each of the compositions prepared in Example 3 and Comparative Examples 5 and 6 as a 1.0% (w/v) aqueous solution, 0.1 μl of the composition was added to a hilltop chamber. Then, the result was attached on the inside of the arms of the 20 testees, and after passing 4 hours, the chamber was removed, and the degree of skin irritation was visually evaluated. The degree of skin irritation was evaluated based on the criteria described in Table 4.

In a common human skin patch test, an aqueous solution diluted to 0.1% is used for 24 hours, however, in this test example, an aqueous solution diluted to 1.0% was used for 4 hours in order to more clearly examine the degree of skin irritation. Table 5 shows the experimental results.

TABLE 4

| Degree of | | Evaluation |
|---|---|---|
| Erythema | Degree of Irritation | Number |
| Strong | Strong Irritation Accompanying | 4 |
| Positive | Swelling and Edema | |
| Medium | Strong Irritation Accompanying | 3 |
| Positive | Edema | |
| Weak Positive | Strong Irritation | 2 |
| False Positive | Weak Irritation | 1 |
| No Side | No Irritation | 0 |
| Effects | | |

TABLE 5

| | | Comparative | Comparative |
|---|---|---|---|
| Subject | Example 3 | Example 5 | Example 6 |
| 1 | 0 | 1 | 0 |
| 2 | 0 | 1 | 1 |
| 3 | 1 | 1 | 1 |
| 4 | 0 | 1 | 1 |
| 5 | 0 | 0 | 0 |
| 6 | 1 | 1 | 1 |
| 7 | 0 | 1 | 0 |
| 8 | 1 | 1 | 1 |
| 9 | 1 | 1 | 1 |
| 10 | 0 | 1 | 1 |
| Average | 0.4 | 0.9 | 0.7 |

Based on the experimental results, it was identified that skin irritation was significantly low in the cosmetic composition of Example 3 compared to Comparative Examples 5 and 6. In Comparative Example 5, highest skin irritation was resulted since octocrylene and avobenzone inducing skin irritation were brought into contact with the skin as they are, and Comparative Example 6 exhibited high irritation compared to Example 3 although the irritation was reduced compared to Comparative Example 5 by using a support. This is considered to be due to the fact that stability of the composite was reduced in the formulation and some of the UV blocker was eluted out of the support.

From the results of Experimental Examples 1 and 2, it was seen that the silica aerogel composite according to the present invention had a high UV blocking effect compared to existing UV blockers even in a small amount, and had low skin irritation, and therefore is suitable to be used in a cosmetic composition for UV blocking.

1. A silica aerogel composite comprising:
hydrophilically surface-modified hydrophobic silica aerogel; and
an organic UV blocker supported in pores of the silica aerogel.

2. The silica aerogel composite of claim 1, wherein the silica aerogel has porosity of 90% or greater, a pore diameter of 2 nm to 10 nm, and a specific surface area of 250 m²/g to 400 m²/g.

3. The silica aerogel composite of claim 1, wherein the silica aerogel has a particle size of 2 μm to 15 μm.

4. The silica aerogel composite of claim 1, wherein the organic UV blocker is one or more types selected from the group consisting of ethylhexyl methoxycinnamate, ethylhexyl silicate, ethylhexyl salicylate, ethylhexyl triazone, octyldimethyl para-aminobenzoic acid, butyl methoxydibenzoylmethane, isoamyl-p-methoxycinnamate, octyl salicylate, octocrylene, butyl methoxydibenzoylmethane, oxybenzone, octyl triazone, menthyl anthranilate, 3,4-methylbenzylidene camphor, bis-ethylhexyloxyphenol methoxyphenyl triazine, homosalate, phenylbenzimidazole sulfonic acid and diethylamino hydroxybenzoyl hexyl benzoate.

5. The silica aerogel composite of claim 1, wherein the organic UV blocker is a mixture of octocrylene and butyl methoxydibenzoylmethane.

6. The silica aerogel composite of claim 1, wherein, in the silica aerogel composite, a content of the organic UV blocker is from 50% by weight to 75% by weight of a total weight of the composite.

7. A cosmetic composition for UV blocking comprising the silica aerogel composite of claim 1.

8. The cosmetic composition for UV blocking of claim 7, wherein the silica aerogel composite is included in 1% by weight to 30% by weight of a total weight of the composition.

9. A cosmetic composition for UV blocking comprising the silica aerogel composite of claim 2.

10. A cosmetic composition for UV blocking comprising the silica aerogel composite of claim 3.

11. A cosmetic composition for UV blocking comprising the silica aerogel composite of claim 4.

12. A cosmetic composition for UV blocking comprising the silica aerogel composite of claim 5.

13. A cosmetic composition for UV blocking comprising the silica aerogel composite of claim 6.
